(19) United States
(12) Patent Application Publication
(10) Pub. No.: US 2004/0043463 A1
(43) Pub. Date: Mar. 4, 2004

(54) Peptide-tagged proteins and compositions for regulating features of the skin or hair; methods of making, and methods of using thereof

(76) Inventor: Pingfan Rao, Fuzhou (CN)

Correspondence Address: Supervisor, Patent Prosecution Services, PIPER RUDNICK LLP, 1200 Nineteenth Street, N.W., Washington, DC, 20036-2412, US

(21) Appl. No.: 10/232,410

(22) Filed: Sep. 3, 2002

(51) Int. Cl. C12N 9/99; C12N 9/02; C12N 9/10; C12N 9/20; C12P 21/04

(52) U.S. Cl. 435/184; 435/189; 435/193; 435/198; 435/69.7

(57) ABSTRACT

A fusion protein containing

(A) an enzyme or enzyme inhibitor; and

(B) a membrane transport sequence,

wherein the enzyme or enzyme inhibitor and the membrane transport sequence are covalently linked at an amino-terminal region, a carboxy terminal region or at any other region of the enzyme or enzyme inhibitor that does not significantly interfere with the enzymatic or inhibitory activity and wherein the enzyme or enzyme inhibitor is a member selected from the group consisting of a tyrosinase, DHICA oxidase (TRP-1), DOPAchrome tautomerase (TRP-2), a superoxide dismutase; a glutathione peroxidase; a lipase, an acetyl-coenzyme A carboxylase, and a fatty acid synthetase. The fusion protein is formulated for topical application to treat hair and skin conditions.

### BACKGROUND OF THE INVENTION

1. Field of the Invention

This invention relates to the field of fusion proteins and the use of protein signal sequences to adapt the delivery of an active agent enzyme or enzyme inhibitor to enhance the treatment or regulation of physical features of the hair and skin. The fusion proteins are applied by topical application in treatments affecting hair pigmentation, skin pigmentation or skin conditioning. All references cited herein are expressly incorporated by reference.

2. Background of the Technology

The topical application of active agents in affecting hair and skin conditions is well known. However, the topical application of active agents for intracellular delivery across the cellular membrane wall for intra-cellular activity has been less evident.

Currently, intracellular delivery is accomplished by utilizing viral vectors or by using non-viral delivery strategies. Historically, non-viral delivery strategies have not been efficient at delivering macromolecules across the cell membrane wall when compared with viral vectors. However, delivery of the active agent across the membrane to enable access to particular intracellular regions of the cell has proven meaningful in modulating cellular activity.

Physical features of the hair and skin reflect the condition of the cells in the dermal and epidermal layers of the skin, including constituent cells such as keratinocytes, follicle cells, melanocytes, adipose cells and others. The regulation of the enzymatic activities within certain cells can offset deterioration in both hair and skin features. Enzymatic regulation is accomplished, by increasing enzyme content or activity to offset any deficit, or by enzymatic inhibition to down-regulate activity along a particular enzymatic pathway. By regulating the pathway, alterations in cellular activity can lead to desirable changes in both hair and skin features.

The skin is a versatile organ that serves as a self-renewing and self-repairing interface between the body of a vertebrate organism and its environment and covers almost the entire external surface of the body. The skin is continuous with, but distinct from, the mucosae of the alimentary, respiratory, and urogenital tracts; the specialized skin of the mucocutaneous junctions connects the skin and the mucosae.

In addition to its protective functions, skin is capable of absorption, and excretion, and is also an important primary site of immunosurveillance against the entry of antigens and initiation of the primary immune response. The skin also performs many biochemical synthetic processes that have both local and systemic effects, and in this sense can be regarded as an endocrine organ. For example, the skin is responsible for the formation of vitamin D, and also synthesizes cytokines and growth factors. For a detailed review of skin and its functions, see Gray's Anatomy: The Anatomical Basis of Medicine and Surgery, Williams et al. (eds.) 1995 Churchill Livingstone, N.Y., pgs. 376-417.

Skin can be divided into two major classes: thin, hairy (hirsute) skin which covers most of the body, and thick, hairless (glabrous) skin which forms the surfaces of the palms of the hands, soles of the feet, and flexor surfaces of the digits. Both classes of skin are composed of three basic layers: the epidermis, the dermis, and the hypodermis. The primary differences in the two classes of skin are in the thickness of their epidermal and dermal components, and in the presence of hairs with their attendant sebaceous glands and arrector pili muscles (pilasebaceous units).

The epidermis, a stratified keratinous squamous epithelium is primarily composed of keratinocytes and can be further divided into several strata (from deep to superficial): stratum basale, stratum spinosum, stratum granulosum, stratum lucidum, and stratum corneum. Epidermal appendages, such as pilosebaceous units, sudoriferous gland, and nails are formed by ingrowth or other modifications of the general epidermis, which is often referred to as the interfollicular epidermis.

In addition to keratinocytes, the mature epidermis also contains nonkeratinocytes including melanocytes which are pigment-forming cells, Langerhans cells which are immunocompetent antigen-presenting cells derived form bone marrow, and lymphocytes. The epidermis also include Merkel cells, which are modified keratinocytes.

The skin, particularly the epidermis, is appealing as a target tissue for delivery of the fusion proteins of the invention. The skin mediates a variety of important local and systemic functions, including maintenance of skin texture, skin color and hair color. These normal skin cell functions can be exploited to modulate the enzymatic pathway associated with lipid generation, hair pigmentation, and removal of free radicals formed as a result of UV exposure.

The skin is an attractive target organ due to its accessibility, thereby providing one of the easiest routes of administration. Moreover, because it is a stratified epithelium, skin allows for the possibility of targeting either differentiated or proliferative cells, depending upon the desired effect of the active agent. In addition, epidermal biology is relatively well-characterized at both the cellular and molecular levels.

It has proven difficult to develop effective methods for importing biologically active molecules into cells, both in vivo and in vitro. Crossing the lipid bilayer has proven to be a significant impediment and no effective means has been developed for the topical application of agents affecting the enzymatic pathways in skin cells. A solution to this problem would greatly expand treatments to skin and hair conditions for which delivery of a biologically active agent to the cell interior would benefit.

In general, conventional non-invasive methods involve pretreatment of the skin to remove hair. However, the more complicated the delivery method or the delivery formulation, the more difficult it is to apply these methods and formulations in the field. Methods that use needles or require multiple dosages via an invasive route meet with problems of patient compliance. In the case of intracellular delivery, it would be desirable to have a means to avoid the use of virus delivery vehicles, which may have undesirable side effects and safety concerns.

Few drugs readily penetrate the intact skin. There is a need in the field for methods of delivery of active agent proteins to within skin cells that does not require special formulations or invasive procedures to facilitate delivery of the protein into skin cells. The present invention addresses this problem.

Hair and Skin Color

Melanogenesis is the process of production and subsequent distribution of melanin by melanocytes within the skin and hair follicles. Melanocytes have specialized lysosome-like organelles, termed melanosomes, which contain several enzymes that mediate the production of melanin. The copper-containing enzyme tyrosinase catalyzes the oxidation of the amino acid tyrosine into DOPA, and subsequently, DOPA-quinone. At least two additional melanosomal enzymes are involved in the eumelanogenesis pathway that produces brown and black pigments, including TRP-1 (DHICA oxidase), and TRP-2 (DOPAchrome tautomerase). Depending on the incorporation of a sulfur-containing reactant, such as cysteine or glutathione, into the products, the melanogenesis pathway diverges to produce the pheomelanins of amber and red pigments.

The perceived color of skin and hair is determined by the ratio of eumelanins to pheomelanins, and in part on blood within the dermis. The balance in skin hue is genetically regulated by many factors, including but not limited to: (a) the levels of expression of tyrosinase, TRP-2, and TRP-1; (b) thiol conjugation (e.g. with glutathione or cysteine) leading to the formation of pheomelanins; (c) the α-melanocyte-stimulating hormone (α-MSH) and melanocortin receptor, which is coupled to the adenylate cyclase/protein kinase A pathway; (d) the product of the agouti locus, agouti signal protein, which down-regulates pigmentation of hair melanocytes; and (e) yet unknown mechanisms that regulate the uptake and distribution of melanosomes in recipient epidermal and hair matrix keratinocytes.

Abnormalities of human skin pigmentation can occur as a result of both genetic and environmental factors. Exposure of the skin (especially Caucasian) to ultraviolet radiation, particularly in the UVB (i.e. intermediate) wavelengths, upregulates synthesis of melanocyte tyrosinase resulting in increased melanogenesis and thus tanning. However, acute or persistent UVB exposure can result in the formation of hyperpigmented lesions or regions of skin, including malignant melanoma skin cancer. Both actinic damage and constitutional abnormalities can produce affected regions such as melasma, age spots, liver spots, freckles and other lentigenes.

Vitiligo is the converse of hyperpigmentation, in which cutaneous melanocytes are either ablated or fail to produce sufficient pigment. Although it would be desirable to restore lost pigmentation in vitiligo-affected skin with topical therapies, this has proven to be quite difficult to accomplish in a high proportion of subjects. As an alternative to pigmentation therapy or cosmetic camouflage with dihydroxyacetone sunless-tanning lotions, one might reduce the normal pigmentation of the unaffected skin to reduce contrast.

Some purportedly “active” or “functional” agents for lightening skin color (e.g. arbutin, kojic acid, niacinamide, licorice, magnesium ascorbyl phosphate, among others) have not been demonstrated yet to be clinically efficacious. The U.S. FDA-approved pharmaceutical products containing 2-4% hydroquinone (“HQ”) are minimally to moderately efficacious. However, HQ has been demonstrated to be cytotoxic to cultured mammalian melanocytes, and mutagenic in Salmonella and mammalian Chinese hamster V79 cells. Hydroquinone's in vitro mechanism of action appears to be primarily a melanocytic cytotoxic effect. Its clinical mechanism of action on whole skin remains uncertain.

Skin Texture/Wrinkling and Acetyl-Coenzyme A (CoA) Carboxylase or Fatty Acid Synthetase

The skin is the largest organ of the body and protects the body from the environmental damage. This protection is provided by the stratum corneum or horny layer of the skin. In this regard, the stratum corneum acts as a barrier (also known as “water barrier” or “permeability barrier”) between the body and the outside environment.

It is now generally accepted that the stratum corneum lipids are the key constituents for a functional barrier. Major classes of stratum corneum lipids include cholesterol, free fatty acids, and ceramides. These lipids are synthesized inside the epidermal cells of the skin and are then secreted into the space between these cells, where they assemble into lamellar bilayer sheets to provide a permeability barrier. The stratum corneum serves as a gate keeper that prevents the entry of infection, chemicals, and other pollutants into the skin. In addition, the stratum corneum prevents the loss of moisture from the skin and thus helps maintain a proper intracellular milieu for normal cellular functions.

In addition to providing a permeability barrier, skin lipids are important for the maintenance of the skin's shape, form, and healthy youthful appearance. Therefore, the skin lipid, its integrity, amount, and the ability to renew itself are crucial for esthetic appearance, such as decreasing wrinkles and other signs of aging. During youth, the blood circulation delivers to the skin all the necessary ingredients for lipid synthesis. However, as we age, the blood flow to the skin decreases. This results in decreased delivery of the lipid building nutrients to the skin. The net result is diminished lipid synthesis and decreased lipid contents of the skin of the aging population.

Depletion and inadequate replenishment of skin lipids leads to moisture loss, dryness, skin wrinkles, and altered appearance. Therefore, restoration of skin's lipid contents is crucial for both health and esthetic reasons. To improve the skin barrier, publications disclose compositions containing natural or synthetic skin lipids. For example, U.S. Pat. No. 5,643,899 discloses the use of lipids for epidermal moisturization and repair of barrier function. However, it is uncertain whether the lipid composition of these products mimic the composition of the human skin lipids. These products contain only from one to three types of lipids, whereas skin lipids are made up of hundreds of types of lipids.

In many instances, lipids in skin care products may have been derived from human and/or animal tissues and thus carry the risk of being contaminated with microorganisms such as viruses and/or bacteria. Furthermore, because lipids in general are unstable, the lipids in these products may undergo peroxidation, and the peroxidation products of lipids may cause harm to the skin. Finally, some exogenous lipids, including ceramides, can actually impede rather than improve the skin's barrier functions. Because of these limitations and concerns about these products, cosmetic compositions which can enhance endogenous production of a correct mix of a full spectrum of physiological lipids by the epidermal cells are highly desirable.

Skin care compositions are known which include some of the compounds disclosed herein. For example, branched-chain amino acids have been employed in skin treatment composition for the treatment of burns, cuts, abrasions, insect bite, dry skin, psoriasis, dermatitis, eczema, and inflammation (U.S. Pat. No. 5,425,954). Sarpotdar, U.S. Pat. No. 4,732,892 discloses a composition for transdermal penetration enhancers containing branched-chain amino acids. Ciavatt, U.S. Pat. No. 4,201,235 discloses a composition for skin, hair, and scalp conditioners containing several amino acids including the branched-chain amino acids. Morelle, U.S. Pat. No. 4,859,653 discloses the use of derivatives of branched-chain amino acids (butyrylvaline and butyrylleucine) for use in treating wrinkling of the human skin.

The role of branched-chain acyl coenzyme A (CoA) to produce fatty acids in the skin was postulated more than 20 years ago (Nicolaides: Science, 186: 19-26, 1974). However, only recently has the incorporation of carbon skeletons of branched-chain amino acids into skin lipids of laboratory animals has been demonstrated (Oku et. al.: Biochim. Biophys. Acta 1214: 279-287, 1994). The art also discloses other compounds individually used in skin care. For example, U.S. Pat. No. 5,472,698 discloses a composition containing lipid building ingredients (serine or its derivatives). However, these ingredients are capable of producing a single class of skin lipids, namely ceramides, and do not include components to produce a full spectrum of skin lipids, namely cholesterol, free fatty acids, and ceramides.

Similarly, skin care compositions are also known to include caprylic acid (also known as octanoate or octanoic acid), either as free acid, but more often in an esterified form as caprylic/capric acid triglycerides. For example, U.S. Pat. No. 5,175,190 discloses a composition for the treatment of skin lesions containing caprylic/capric triglycerides. U.S. Pat. No. 5,569,461 discloses a topical antimicrobial composition containing a monoester of caprylic acid. U.S. Pat. No. 4,760,096 discloses a moisturizing skin preparation containing caprylic/capric acid triglycerides. U.S. Pat. No. 4,495,079 discloses a composition for facial skin cleanser capable of softening and removing sebum plaque containing a mixture of caprylic acid and capric acid esterified to a fatty alcohol. U.S. Pat. No. 5,472,698 discloses the use of several thiol compounds, including the use of lipoic acid in enhancing lipid production in the skin. There remains a need, however, for compositions and methods that among other things increase lipid production in the skin.

Acetyl-CoA carboxylase and fatty acid synthetase are the two major enzymes involved in the synthesis of fatty acids in animals. The activities of both enzymes are affected by nutritional manipulations. Although acetyl-CoA carboxylase is considered generally to be the rate-limiting step in lipogenesis, there is evidence that suggests that fatty acid synthetase may become rate limiting under certain conditions.

The principal support for the view that acetyl-CoA carboxylase is the rate-limiting enzyme for lipogenesis is that the activity of the enzyme is controlled by allosteric effectors that change the catalytic efficiency of the enzyme. Fatty acid synthetase appears to be subject to the type of control necessary for an enzyme to serve as a regulator of the rate of a biological process over a short term.

Skin/Hair Color and Tyrosinase

Tyrosinase is the key enzyme for melanin biosynthesis. Disorders of tyrosinase activity include Parkinson's disease, vitellego and albinism. As described herein, this invention provides a method of modifying tyrosinase to enhance intracellular melanin production.

Thus, the invention provides uniquely effective protocols and materials for the treatment of disorders related to tyrosinase activity. In particular, the invention provides protocols and materials for treating pigmentation disorders by enhancing the availability tyrosinase in hair follicle cells.

The treatment of the hair and skin with various creams or lotions with biologically active ingredients to improve hair growth and pigmentation has generally been unsatisfactory. A wide variety of externally applied agents available are said to improve body, flexibility, curl and hair color. These have limited and only short term usefulness. In particular, coloring hair with various dyes requires frequent repetitions and is not always natural in appearance. The invention provides improved alternatives, focused on tyrosinase.

Tyrosinase is a ubiquitously distributed copper-containing monoxygenase that is essential for melanin biosynthesis in pigment cells. It catalyzes the conversion of tyrosine to dihydroxyphenylalanine (DOPA) and the conversion of DOPA to dopaquinone, referred to as tyrosine hydroxylase activity and DOPA oxidase activity, respectively.

Disorders of tyrosinase expression and melanin biosynthesis are related to many diseases involving pigmentation such as albinism, hair pigment loss, and vitellego. Tyrosinase is a key enzyme for melanin synthesis in vertebrate pigment cells, melanocytes, and retinal pigment epithelial cells. Tyrosinase is absent in human white hair bulbs, as well as in albino epithelial cells. Thus, the loss of tyrosinase could be the basis of pigment loss in hair.

Generally, the present invention is also directed to compositions and methods for treating disorders related to tyrosinase activity and melanin biosynthesis. In another aspect, the invention is directed to a method for treating a tyrosinase deficiency or pigmentation disorder in a subject which method comprises modifying the content of tyrosinase in cells by enhancing delivery of the tyrosinase according to the invention.

In a preferred embodiment, the cells to be treated are hair follicle cells and the subjects are mammals, such as primates, including humans. The topical delivery of the modified tyrosinase can be utilized as the sole component in the method of treatment, but the modified tyrosinase may also be used in combination with administration of other beneficial compounds such as proteins, pigments, dyes, growth regulators and other compounds which affect the characteristics of skin and hair.

Skin Condition and Superoxide Dismutase

Active oxygen liberated in a living body must be rapidly consumed. Otherwise, various cell elements such as DNA, lipids and proteins become the target molecules for oxidation, and breakdown of the functions of the cells accompanies the production of lipid peroxides. The body possesses systems for the elimination of these active oxygens, of which superoxide dismutase (SOD), catalase, and glutathione peroxidase (GSH-Px) are known.

SOD has attracted much attention as a catalyst for decomposing and detoxifying superoxides, thus lowering the amount of lipid peroxides (LPO) in the epidermis due to ultraviolet rays, when SOD is applied externally to the skin (R. Ogura et. al., The Biological Role of Reactive Oxygen Species in Skin, edited by O. Hayaishi, S. Imamura and Y. Miyachi, University of Tokyo Press, 1987, p. 55).

Intravenously injected SOD derivatives prevent or considerably alleviate cerebral ischemic disorders, myocardial ischemic disorders, acute gastric mucosal disorders, carrageenin edema, hemorrhagic shock, cerebral edema, renal ischemic disorders, etc. (M. Inoue and N. Watanabe: “Antioxidants in Therapy and Preventive Medicine,” edited by I. Emerit, Plenum Press, 1990).

Signal Peptides

Signal peptide sequences guide the translocation of most intracellular secretory proteins across the endoplasmic reticulum (ER) and plasma membranes through protein-conducting channels. Secretory protein transport also support a role for the signal sequence in targeting proteins to certain cellular membranes (B. Alberts et. al., Molecular Biology of the Cell, Third Edition, Garland Publishing, 1994, pp. 557-585).

Several types of signal sequence-mediated translocation pathways from have been proposed for exiting from the interior of the membrane. The major model implies that the proteins are transported across membranes through a hydrophilic protein-conducting channel formed by a number of membrane proteins.

In eukaryotic cells, newly synthesized proteins in the cytoplasm are targeted to the ER membrane by signal sequences that are recognized generally by the signal recognition particle (SRP) and its ER membrane receptors. This targeting step is followed by the actual transfer of protein across the ER membrane and out of the cell through the protein-conducting channel. In bacteria, the transport of most proteins across the cytoplasmic membrane also requires a similar protein-conducting channel. On the other hand, signal peptides can interact strongly with lipids, so transport of some secretory proteins across cellular membranes can occur directly through the lipid bilayer in the absence of any proteinaceous channels.

It has proven difficult to develop effective methods for importing biologically active molecules into cells, both in vivo and in vitro. Crossing the lipid bilayer has proven to be a significant impediment and no effective means has been developed for the topical application of agents affecting the enzymatic pathways in skin cells. A solution to this problem would greatly expand treatments to skin and hair conditions for which delivery of a biologically active agent to the cell interior would benefit.

The present invention solves this problem by providing a method of importing a biologically active molecule into a cell using mechanisms naturally occurring in cells and thus avoiding damaging the target cells. Additionally, the present method can be used to import molecules into large numbers of cells upon topical application to the skin exterior and employed in the treatment of numerous skin and hair conditions.

Localization Signal Peptides for Fusion Proteins in Active Agent Delivery

The present invention provides the discovery that importing exogenous biologically active fusion proteins into the cells of the epidermis or dermis can be accomplished in forming a fusion protein by covalently linking an importation competent signal peptide sequence to a selected biologically active enzyme or enzyme inhibitor protein and administering the fusion protein to the cell by topical application onto the skin. The complex is then imported across the cell membrane by the cell. Thus, the present invention provides a method of importing a fusion protein into an epidermal cell by topical application of the active agent fusion protein within a cream, ointment or tonic.

Specific targeting of tissues or cells with peptides depends on the presence of unique or differentially expressed markers on cells. The plasma membrane of eukaryotic cells is the first barrier which must be traversed by agents acting on intracellular targets. In the detailed description that follows, certain specific sequences have been identified according to the invention that can expedite transport when fused to the active agent enzymes and inhibitors identified above.

### SUMMARY OF THE INVENTION

The present invention meets the above-identified need by providing fusion proteins in compositions and methods for modulating enzymatic activity in skin cells, involving administering a fusion protein comprising an enzyme or enzyme inhibitor linked to at least one signal peptide.

More particularly, the invention relates to the modification of proteins or enzymes affecting hair pigmentation, skin pigmentation or skin conditioning.

A protein transduction sequence enhances the intracellular delivery of the enzyme or protein to the desired targeted local region of the cell in which the protein or enzyme acts to effect the desired change in a subject.

The fusion proteins are prepared by chemical modification of the active agent or by recombinant production of a fusion protein containing both the active agent protein and a signal peptide sequence. Fusion proteins of the invention are applied topically on the skin of the subject, and absorbed into the cells of the lower skin strata through intracellular transduction at a higher rate than compared to topical application of the active agent enzyme or protein alone. Thus, the present invention unexpectedly provides for effective topical application for intracellular delivery, without the disadvantages of oral or parental administration.

In contrast, the prior art appears to comprehend only that topical application of ordinary enzymes or protein can promote some small changes in affecting intracellular activity along an enzymatic pathway, but not all the enzymes comprehended by the invention and no such enzymes in combination with an intracellular transmembrane signaling sequence. In addition, enzyme inhibition by delivery of an inhibitor is not taught or suggested anywhere in the prior art. Thus, the present invention also includes a method where the administration of fusion protein results in a decreased enzymatic activity.

While not being bound by any particular theory of how covalent linking of signal peptide(s) to the enzyme or enzyme inhibitor enhances transmembrane delivery, linkage can occur at the amino-terminal region or carboxy-terminal region of the protein or at any site along the protein structure that will not significantly interfere with the active agent protein as an enzyme or enzyme inhibitor.

Other features and advantages of the present invention will be readily appreciated as the same becomes better understood by reference to the following detailed description.

### DETAILED DESCRIPTION OF THE INVENTION

The potential advantages of fusion proteins in methods of treatment of the hair and skin by topical administration are appreciated, but have not been achieved by methods of the prior art. The present invention includes methods for making and using fusion protein compositions for preventing or treating skin and hair deficiencies in a mammal, preferably a human subject, which offer improved efficacy of delivery of the active agent protein to within the cell via a membrane transport sequence.

The intracellular action of the enzyme or enzyme inhibitor is known to play a critical role in regulating cellular activity in affecting, for example, skin and hair conditions. The present invention succeeds in regulating intracellular activity by delivery of the fusion protein through topical application and then inducing the desired cellular response in a mammal, to improve skin and hair conditions.

Short cellular sequences capable directing the movement of a “cargo” enzyme or protein have now been identified. These sequences function either via endocytic pathways or through a proposed mechanism referred to as ‘inverted micelles.’ Based upon their amino acid sequence, all known import signals can be broadly classified as either hydrophobic, amphipathic or cationic.

The specific import signals utilized according to the invention are as follows:

| | | |
|---|---|---|
| IMPORT SIGNAL | SOURCE | AMINO ACID SEQUENCE |

Hydrophobic sequences

| | | |
|---|---|---|
| Membrane Permeable | Karposi FGF | AAVALLPAVLLALLAP |
| Sequences (MPSs) | Grb2 (SH2 domain) | AAVLLPVLLAAP |
| | Integrin β3 | VTVLALGALAGVGVG |
| Fusion sequence | HIV-1 GP41(1-23) | GALFLGFLGAAGSTMGA |
| Signal sequence | Caiiman croc. | MGLGLHLLVLAAALQGAMGLGL |
| | Lg(v) light chain | HLLLAAALQGA |

Amphipathic/Cationic Sequences

| | | |
|---|---|---|
| KALA | Influenza HA-2 (1-20) | WEAKLAKALAKALAKH |
| | | LAKALAKALKACEA |
| GALA | | WEAALAEALAEALAEHLAEA |
| | | LAEALEALAA |
| 4₆ | | LARLLARLLARLLRALLRALLRAL |
| HEL 11-7 | | KLLKLLLKLWKLLLKLLK |
| Penetratin or Antp | Antennapedia | RQIKIWFQRRMKKWK |
| | third helix (43-58) | |
| Tat | HIV-1 Tat (47-57) | YGRKKRRQRRR |
| VP22 | HSV transcription | DAATATRGRSAASRPTERPRA |
| | factor (267-300) | PARSASRPRRPVE |
| Transportan | Galanin + Mastoparan | GWTLNSAGYLLGKINLKALAALAKKIL |

One group of hydrophobic sequences called membrane permeable sequences (MPSs) is derived from the hydrophobic region of various signal sequences. MPSs adopt a characteristic α-helical conformation under membrane mimetic environments, despite the lack of primary sequence homology between the signal sequences. These hydrophobic regions can be from about 18 to 21 amino acids long. They traverse the cell membrane and are therefore able to import covalently attached functional domains from other intracellular proteins. Examples of such domains include the src homology 2 (SH2) domain of Grb2, human integrin proteins β₁, β₃ and α_{hb} and the Nuclear Localization Signal (NLS) of NFκB p50. Other hydrophobic signal sequences [HIV gp41 fusion peptide, Caiiman crocodylus immunoglobulin (v) light chain signal sequence] have also been fused to the NLS sequence derived from the SV40 large T antigen to target the nucleus of cells and deliver antisense oligonucleotides and plasmid DNA.

Amphipathic sequences harbor a periodicity of hydrophobic and polar residues. These sequences, typified by the fusion peptide of influenza hemagluttinin (HA-2) and related synthetic analogs [GALA, KALA, 4₆ and Hel 11-7] represent a group of import signals that have been shown to interact with cellular membranes. Their interaction with the uncharged lipid bilayers results in fusion events with the membrane. The lower pH present in vesicles causes these sequences to undergo a random coil to α-helical transition that induces leakage of vesicular contents. The peptides 4₆ and Hel 11-7 have been shown to transport plasmid DNA into adherent cell lines.

Cationic peptide sequences represent the final group of import signals. Polylysine sequences have been used for several decades as a method of importing various macromolecules across the cell membrane. These sequences interact with the negatively charged phospholipids of the cell membrane and enter the cell via the endocytic pathway. Penetratin from the third helix of the Antp and Transportan created from the fusion of galanin to mastoparan sequences, penetrate cell membranes via a postulated inverted micelle pathway.

These signal sequences, when coupled to an enzyme or protein cargo sequence form fusion proteins for transport into the intracellular regions of the skin cells that then modulate the enzymatic pathways associated with hair and skin conditions. The fusion peptides according to the invention are demonstrated in the following examples.

Definitions

Peptide. The term “peptide” is used herein interchangeably with “oligopeptide” to designate a series of monomers or residues, typically L-amino acids, connected one to the other typically by peptide bonds between the alpha-amino and carbonyl groups of adjacent amino acids. The term peptide encompasses an isolated or recombinant sequence of amino acids, which may be naturally occurring or non-naturally occurring, and synthetic derivatives or analogues thereof. Sequences of naturally occurring amino acids recited herein utilize the standard amino acid nomenclature using single letter abbreviations for each residue—Alanine (A), Arginine (R), Asparagine (N), Aspartic acid (D), Cysteine (C), Glutamine (Q), Glutamic acid (E), Glycine (G), Histidine (H), Isoleucine (I), Leucine (L), Lysine (K), Methionine (M), Phenylalanine (F), Proline (P), Serine (S), Threonine (T), Tryptophan (W), Tyrosine (Y), Valine (V). Amino acid “analogues” encompass functionally equivalent modified amino acid residues which are known in the art (see, e.g., U.S. Pat. Nos. 5,221,665 and 6,171,589, both incorporated by reference).

The term “membrane transport sequence” or “MTS” is used to indicate a peptide, or derivative thereof, that directs the transport of a peptide, protein, or molecule associated with the MTS; from the outside of a cell into the cytoplasm of the cell through a cytoplasmic membrane of the cell. Furthermore, a peptide that contains a “membrane transport sequence” and additional amino acid sequences could be used as a “membrane transport sequence” for the purposes of the present invention. An MTS may be composed of D- or L-amino acids.

The term “nuclear localization sequence” or “NLS” is used to indicate a peptide, or derivative thereof, that directs the transport of a peptide, protein, or molecule associated with the NLS; from the cytoplasm into the nucleus of the cell across the nuclear membrane. Furthermore, a peptide that contains a “nuclear localization sequence” and additional amino acid sequences could be used as a “nuclear localization sequence” for the purposes of the present invention Adam et al. (1990) J. Cell. Biol. 11 1:807-818). In certain embodiments, an NLS may be composed of D- or L-amino acids.

Formulations, Dosage and Administration

Fusion proteins of the invention may be formulated in compositions for delivery via an appropriate route using formulations known in the art for other topical applications, for instance, as described in various U.S. patents cited herein. Those skilled in the art will appreciate that the disclosed compositions of the present invention are aqueous or non-aqueous preparations for administration to mammals, and preferably humans.

### EXAMPLES

The following examples are put forth so as to provide those of ordinary skill in the art with a complete disclosure and description of how to carry out the invention and are not intended to limit the scope of what the inventors regard as their invention, nor are they intended to represent or imply that the experiments below are all of or the only experiments performed. Efforts have been made to ensure accuracy with respect to numbers to be used (e.g., amounts, temperatures, etc.), but some experimental error and deviation should be accounted for. Unless indicated otherwise, parts are parts by weight, molecular weight is weight average molecular weight, and temperature is in degrees Centigrade.

### Example 1

Tat-GFP Fusion Protein in Assessing Intracellular Distribution

Preparation of the fusion protein. GFP (Green Fluorescent Protein) is a marker for illustrating the distribution of a protein composition in a cell population sample and can demonstrate intracellular delivery across the cell membrane in a fusion protein according to the invention. A TAT-GFP fusion protein was constructed to investigate the distribution in different skill cells when applied directly the skin of model subject mammals.

### Example 2

Tat-Tyrosinase Fusion Protein Preparation and Method of Enhancing Pigmentation of Hair and Skin

A Tat-Tyrosinase fusion protein can be produced by chemical synthesis or by recombinant method. The fusion protein can have the Tat signal sequence covalently attached at the amino-terminal region, the carboxy-terminal region or at any other region of the enzyme, so long as the covalently attached Tat signal sequence which will not significantly interfere with the activity of the Tyrosinase enzyme.

A fusion protein thus prepared can be formulated in a cream or ointment for topical application to the skin to enhance pigmentation of the skin or hair in cells proximate to the application area. In another embodiment, a Tat-Tyrosinase fusion protein of this invention is applied or administered to the skin during an appropriate period and using a sufficient number of dosages to achieve enhanced skin pigmentation. The concentration of active agent in the composition will depend on absorption, inactivation, and excretion rates of the compound as well as other factors known to those of skill in the art.

It is to be noted that dosage values will also vary with the severity of the condition to be treated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered as a single dose, or may be divided into a number of smaller doses to be administered at varying intervals of time.

Topical and other formulations of the Tat-Tyrosinase fusion protein are of utility in enhancing skin or hair pigmentation in humans and other animals. These formulations may be useful for pure cosmetic purposes, simply to obtain a darker skin color for perceived beautification.

The compounds of this invention act primarily by increasing mammalian melanocyte tyrosinase, the rate-limiting enzyme in the production of melanin from tyrosine and DOPA. If desirable these formulations could also be used to increase pigmentation in hair, albeit during the biosynthesis of hair, by enhancing pigment production within the melanocytes of hair follicles. The formulations would likely not affect the already emerged pigmented portions of hair, unlike a coloring agent.

The formulations useful in the present invention contain biologically effective amounts of the Tat-Tyrosinase fusion protein. A biologically effective amount of the active agent is understood by those skilled in the art to mean that a sufficient amount of the agent in the composition is provided such that upon administration to the human or animal by topical route, sufficient active agent is provided on each application to give a desired result. However, the biologically effective amount of the active compound is at a level that it is not toxic to the human or animal during the term of treatment. By a suitable biologically compatible carrier, when the fusion protein is topically applied, it is understood that the carrier may contain any type of suitable excipient in the form of cosmetic compositions, pharmaceutical adjuvants, lotions, creams, and the like. In one embodiment the active agent is administered in a liposomal carrier. The active agent is administered for a sufficient time period to enhance the desired symptoms and the clinical signs associated with the condition being treated, or to achieve the level of desired skin or hair pigmentation. The individual dosage, dosage schedule, and duration of treatment may be determined by clinical evaluations by those of skill in the art.

Solutions or suspensions for topical application can include the following components: a sterile diluent such as water, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediamine-tetraacetic acid (EDTA); buffers such as acetates, citrates or phosphates; and agents for the adjustment of tonicity such as sodium chloride or dextrose. The pH can be adjusted with acids or bases, such as hydrochloric acid or sodium hydroxide.

Suitable vehicles, carriers, or formulations for topical application are known, and include lotions, suspensions, ointments, oil-in-water emulsions, water-in-oil emulsions, creams, gels, tinctures, sprays, powders, pastes, and slow-release transdermal or occlusive patches. Thickening agents, emollients, and stabilizers can be used to prepare topical compositions. Examples of thickening agents include petrolatum, beeswax, xanthan gum, or polyethylene glycol, humectants such as sorbitol, emollients such as mineral oil, lanolin and its derivatives, or squalene. A number of solutions and ointments are commercially available, especially for dermatologic applications.

The fusion proteins can be provided in the form of pharmaceutically-acceptable salts. As used herein, the term “pharmaceutically-acceptable salts or complexes” refers to salts or complexes that retain the desired biological activity of the parent compound and exhibit minimal, if any, undesired toxicological effects. Examples of such salts are (a) acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acids, naphthalenedisulfonic acids, and polygalacturonic acid; (b) base addition salts formed with polyvalent metal cations such as zinc, calcium, bismuth, barium, magnesium, aluminum, copper, cobalt, nickel, cadmium, and the like, or with an organic cation formed from N,N-dibenzylethylene-diamine or ethylenediamine; or (c) combinations of (a) and (b); e.g., a zinc tannate salt or the like.

The fusion proteins can be modified in order to enhance their usefulness as pharmaceutical compositions. For example, it is well know in the art that various modifications of the active agent, such as alteration of charge, can affect water and lipid solubility and thus alter the potential for percutaneous absorption. The vehicle, or carrier, can also be modified to enhance cutaneous absorption, enhance the reservoir effect, and minimize potential irritancy or neuropharmacological effects of the composition.

Thus, the invention provides various formulations of Tat-Tyrosinase and other fusion proteins as topical skin or hair pigment enhancers containing the active agents described above. The invention further provides formulations as topical anti-oxidants containing the active agent fusion protein and/or functional compounds described above. Such formulations can be made in combination with other active and/or functional ingredients used in skincare products (e.g. organic or inorganic lotion, antioxidant, anti-inflammatory, anti-erythema, antibiotic, antimicrobial, humectant, or other ingredients). Other ingredients can be formulated with the fusion proteins to augment their effect, including but not limited to Vitamin C, Vitamin E, magnesium ascorbyl phosphate, aloe vera extract, and retinoic acids. In addition, alpha-hydroxy acids can be included to speed up the skin pigmentation process by exfoliating surface skin.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

We claim:

1. A fusion protein containing:
(A) an enzyme or enzyme inhibitor; and
(B) a membrane transport sequence,
wherein the enzyme or enzyme inhibitor and the membrane transport sequence are covalently linked at an amino-terminal region, a carboxy terminal region or at any other region of the enzyme or enzyme inhibitor that does not significantly interfere with the enzymatic or inhibitory activity and wherein the enzyme or enzyme inhibitor is a member selected from the group consisting of a tyrosinase, DHICA oxidase (TRP-1), DOPAchrome tautomerase (TRP-2), a superoxide dismutase; a glutathione peroxidase; a lipase, an acetyl-coenzyme A carboxylase, and a fatty acid synthetase.

2. The fusion protein according to claim 1, wherein the membrane transport sequence is a member selected from the group consisting of:
AAVALLPAVLLALLAP,
AAVLLPVLLAAP,
VTVLALGALAGVGVG,
GALFLGFLGAAGSTMGA,
MGLGLHLLVLAAALQGAMGLGL,
HLLLAAALQGA,
WEAKLAKALAKALAKH,
LAKALAKALKACEA,
WEAALAEALAEALAEHLAEA,
LAEALEALAA,
LARLLARLLARLLRALLRALLRAL,
KLLKLLLKLWKLLLKLLK,
RQIKIWFQRRMKKWK,
YGRKKRRQRRR,
DAATATRGRSAASRPTERPRA,
PARSASRPRRPVE,
GWTLNSAGYLLGKINLKALAALAKKIL, and
a polylysine.

3. A composition comprising a fusion protein containing:
(A) an enzyme or enzyme inhibitor; and
(B) a membrane transport sequence,
wherein the enzyme or enzyme inhibitor and the membrane transport sequence are covalently linked at an amino-terminal region, a carboxy terminal region or at any other region of the enzyme or enzyme inhibitor that does not significantly interfere with the enzymatic or inhibitory activity and wherein the enzyme or enzyme inhibitor is a member selected from the group consisting of a tyrosinase, DHICA oxidase (TRP-1), DOPAchrome tautomerase (TRP-2), a superoxide dismutase; a glutathione peroxidase; a lipase, an acetyl-coenzyme A carboxylase, and a fatty acid synthetase.

4. The composition according to claim 3, wherein the membrane transport sequence is a member selected from the group consisting of:
AAVALLPAVLLALLAP,
AAVLLPVLLAAP,
VTVLALGALAGVGVG,
GALFLGFLGAAGSTMGA,
MGLGLHLLVLAAALQGAMGLGL,
HLLLAAALQGA,
WEAKLAKALAKALAKH,
LAKALAKALKACEA,
WEAALAEALAEALAEHLAEA,
LAEALEALAA,
LARLLARLLARLLRALLRALLRAL,
KLLKLLLKLWKLLLKLLK,
RQIKIWFQRRMKKWK,
YGRKKRRQRRR,
DAATATRGRSAASRPTERPRA,
PARSASRPRRPVE,
GWTLNSAGYLLGKINLKALAALAKKIL, and
a polylysine.

5. A method of making a fusion protein comprising:
combining an enzyme or enzyme inhibitor with a membrane transport sequence, wherein the enzyme or enzyme inhibitor and the membrane transport sequence are combined in any order and covalently linked at an amino-terminal region, a carboxy terminal region or at any other region of the enzyme or enzyme inhibitor that does not significantly interfere with the enzymatic or inhibitory activity and wherein the enzyme or enzyme inhibitor is a member selected from the group consisting of a tyrosinase, DHICA oxidase (TRP-1), DOPAchrome tautomerase (TRP-2), a superoxide dismutase; a glutathione peroxidase; a lipase, an acetyl-coenzyme A carboxylase, and a fatty acid synthetase.

6. The method according to claim 5, wherein the membrane transport sequence is a member selected from the group consisting of:
AAVALLPAVLLALLAP,
AAVLLPVLLAAP,
VTVLALGALAGVGVG,
GALFLGFLGAAGSTMGA,
MGLGLHLLVLAAALQGAMGLGL,
HLLLAAALQGA,
WEAKLAKALAKALAKH,
LAKALAKALKACEA,
WEAALAEALAEALAEHLAEA,
LAEALEALAA,
LARLLARLLARLLRALLRALLRAL,
KLLKLLLKLWKLLLKLLK,
RQIKIWFQRRMKKWK,
YGRKKRRQRRR,
DAATATRGRSAASRPTERPRA,
PARSASRPRRPVE,
GWTLNSAGYLLGKINLKALAALAKKIL, and
a polylysine.

7. The method of claim 5, wherein the fusion protein is made by chemical synthesis.

8. The method of claim 5, wherein the fusion protein is made by recombinant method.

9. A method of treatment comprising:
applying to a topical surface of a subject a fusion protein containing
(A) an enzyme or enzyme inhibitor; and
(B) a membrane transport sequence,
wherein the enzyme or enzyme inhibitor and the membrane transport sequence are covalently linked at an amino-terminal region, a carboxy terminal region or at any other region of the enzyme or enzyme inhibitor that does not significantly interfere with the enzymatic or inhibitory activity and wherein the enzyme or enzyme inhibitor is a member selected from the group consisting of a tyrosinase, DHICA oxidase (TRP-1), DOPAchrome tautomerase (TRP-2), a superoxide dismutase; a glutathione peroxidase; a lipase, an acetyl-coenzyme A carboxylase, and a fatty acid synthetase.

10. The method according to claim 9, wherein the membrane transport sequence is a member selected from the group consisting of:
AAVALLPAVLLALLAP,
AAVLLPVLLAAP,
VTVLALGALAGVGVG,
GALFLGFLGAAGSTMGA,
MGLGLHLLVLAAALQGAMGLGL,
HLLLAAALQGA,
WEAKLAKALAKALAKH,
LAKALAKALKACEA,
WEAALAEALAEALAEHLAEA,
LAEALEALAA,
LARLLARLLARLLRALLRALLRAL,
KLLKLLLKLWKLLLKLLK,
RQIKIWFQRRMKKWK,
YGRKKRRQRRR,
DAATATRGRSAASRPTERPRA,
PARSASRPRRPVE,
GWTLNSAGYLLGKINLKALAALAKKIL, and
a polylysine.
